(19) United States
(12) Patent Application Publication
(10) Pub. No.: US 2025/0228691 A1
(43) Pub. Date: Jul. 17, 2025

(54) COMPRESSION ASSEMBLY TO ALLEVIATE PHYSICAL DISCOMFORT

(71) Applicant: ZIIVAA IP, LLC, SARASOTA, FL (US)

(72) Inventors: Robert BRADY, Sarasota, FL (US); Jennifer JENSEN, University Park, FL (US)

(21) Appl. No.: 19/021,847

(22) Filed: Jan. 15, 2025

Related U.S. Application Data
(60) Provisional application No. 63/620,930, filed on Jan. 15, 2024.

(51) Int. Cl. A61F 5/02

(52) U.S. Cl. CPC A61F 5/028

(57) ABSTRACT

A compression assembly to alleviate physical discomfort includes a fabric material having a waist opening on a first end and two leg openings on an opposing second end. The also assembly includes a tensioning device secured to a portion of the fabric material, and a plurality of guides integrated with the fabric material. The plurality of guides run in a latitudinal direction along the fabric material between the waist opening and the two leg openings. In addition, the compression assembly includes a plurality of tensioning elements positioned within the plurality of guides, with each tensioning element having a distal end anchored to the fabric material and a proximal end secured to the tensioning device. The tensioning device is configured to selectively shorten a length of the plurality of tensioning elements and result in applying compression to hip and back areas of a user wearing the compression assembly.

### CROSS-REFERENCE TO RELATED APPLICATIONS

This application claims the benefit of U.S. provisional patent application 63/620,930 filed Jan. 15, 2024, which is hereby incorporated herein in its entirety.

### TECHNICAL FIELD

The present invention relates to the field of compression devices to alleviate discomfort associated with physical and medical conditions.

### BACKGROUND

Compression can help alleviate discomfort associated with various physical conditions by providing pain relief, support, and stabilization, which in turn may improve circulation, reduce swelling, reduce the risk of blood clots, enhancing healing, prevent injuries or enhance the recovery process. Compression can also alleviate discomfort associated with the menstrual cycle or with discomfort associated with physical conditions such as dysmenorrhea, endometriosis, sciatica nerve pain, arthritis, tendonitis, lower back pain, etc.

Existing compression garments require wrapping several layers around the body in complex patterns. Other compression garments require additional components that may result in an unpleasant look, i.e., making it obvious that the device is being worn and/or signaling that the wearer is suffering from a potentially private physical condition. Many compression devices require assistance from another person to position properly. This process can be inconvenient, uncomfortable, and time consuming. Additionally, the level of compression may be difficult to customize and control according to personal needs and preferences.

Therefore, there is a need in the industry to provide relief to a wearer that would also improve the experience in terms of convenience and self-adjustability by the wearer, as well as a potentially discrete non-obvious appearance under normal attire, simplicity, convenience, comfort, and customizability throughout the day.

### SUMMARY

A compression assembly to alleviate physical discomfort is disclosed. The compression assembly includes a fabric material having a waist opening on a first end and two leg openings on an opposing second end. The assembly also includes a tensioning device secured to a portion of the fabric material, and a plurality of guides integrated with the fabric material. The plurality of guides run in a latitudinal direction along the fabric material between the waist opening and the two leg openings, and a plurality of tensioning elements are positioned within the plurality of guides with each tensioning element having a distal end anchored to the fabric material and a proximal end secured to the tensioning device.

The tensioning device is configured to selectively shorten a length of the plurality of tensioning elements and result in applying compression to hip and back areas of a user wearing the compression assembly. In addition, the plurality of guides may include a first set of guides and a second set of guides, where the first set of guides are positioned to run at least partially around a perimeter of the fabric material in a first direction, and the second set of guides are positioned to run at least partially around the perimeter of the fabric material in an opposing second direction. Likewise, the plurality of tensioning elements may include a first set of tensioning elements and a second set of tensioning elements, where the first set of tensioning elements are positioned to run through the first set of guides in the first direction, and the second set of tensioning elements are positioned to run through the second set of guides in the opposing second direction. The plurality of tensioning elements may include inelastic cables or elastic bands.

The tensioning device may include a first tensioning device and a second tensioning device, where the first tensioning device is secured to respective proximal ends of the first set of tensioning elements, and the second tensioning device is secured to respective proximal ends of the second set of tensioning elements. The plurality of tensioning elements may include inelastic cables or elastic bands.

The fabric material may be sized and shaped to apply compression to hip and back areas of a user in a first stage of initial compression, and the tensioning device may be configured to apply the compression to the hip and back areas of the user in a second stage of compression.

The tensioning device may include a rotating compression adjustment knob to selectively wind the plurality of tensioning elements thereon to maintain and apply a selected amount of compression in the second stage of compression. The tensioning device may also have a ratcheting mechanism to maintain tension in the plurality of tensioning elements and a release mechanism that disengages the ratcheting mechanism when activated. The tensioning device may be positioned on a rear, side, or front portion of the fabric material.

In another aspect, the compression assembly includes a compression band having a waist opening and is configured to be worn around a hip area by a user. A tensioning device is secured to a portion of the compression band, a guide is secured to the compression band and runs at least partially in a latitudinal direction along the compression band, and a tensioning element is positioned within the guide and having a distal end anchored to the compression band and a proximal end secured to the tensioning device. The tensioning device is configured to selectively shorten a length of the tensioning element and result in applying compression to a user wearing the compression assembly. The distal end of the tensioning element also may be formed into an adjustable loop that is coupled back on itself.

In another aspect, a method of applying compression to a hip area of a user using the compression assembly described above is disclosed. The method includes activating the tensioning device to selectively shorten a length of the tensioning element and result in applying compression to the user wearing the compression band.

The method also includes activating the tensioning device to selectively lengthen the tensioning element and result in reducing an amount of compression being applied to the user wearing the compression band.

### BRIEF DESCRIPTION OF THE DRAWINGS

The aspects and the attendant advantages of the embodiments described herein will become more readily apparent by reference to the following detailed description when taken in conjunction with the accompanying drawings wherein:

FIGS. 1A, 1B, and 1C are views of a compression assembly to alleviate discomfort in which various aspects of the disclosure may be implemented;

FIG. 2 is a side of the compression assembly of FIG. 1B having an attachment mechanism to secure the compression assembly to the wearer;

FIG. 3 is a rear view of the compression assembly of FIG. 1C having a tensioning device positioned on a rear portion;

FIGS. 4A, 4B, and 4C are views of the compression assembly having wider compression field in another aspect;

FIGS. 5A, 5B, and 5C are views of the compression assembly having a larger disbursement of tensioning elements in another aspect;

FIGS. 6A, 6B, and 6c are views of the compression assembly having a serpentine configuration in another aspect;

FIGS. 7A, 7B, and 7C are views of the compression assembly having a modified serpentine configuration in another aspect;

FIGS. 8A, 8B, and 8C are views of the compression assembly having the tensioning device positioned on a front portion in another aspect;

FIGS. 9A and 9B are views of the compression assembly having two sets of tensioning elements in yet another aspect;

FIGS. 10A and 10B are views of the compression assembly having a pneumatic compression band for applying compression; and

FIGS. 11A and 11B are views of the compression band.

### DETAILED DESCRIPTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

The present invention is directed to a compression assembly that may be worn by a user to alleviate discomfort, i.e., pain, tightness, soreness, etc., associated with a predetermined condition by applying user adjustable compression to the perimeter of the hips and/or hip area, lower back and/or lower abdomen areas of the user. For example, the present invention alleviates discomfort associated with the menstrual cycle or with discomfort associated with physical conditions such as dysmenorrhea, endometriosis, sciatica nerve pain, arthritis, tendonitis, lower back pain, etc.

As contemplated herein, a predetermined condition may comprise a physical or medical condition. The inventive compression assembly is intended to be worn as clothing outerwear or discretely under clothing and should be sufficiently flexible to allow for movement, adjustment, and an appropriate fit range among users. It is within the scope of the present invention that wearing the compression assembly should not substantially impact mobility or flexibility.

The compression assembly generally comprises a compression garment and an integrated compression band. However, in some embodiments, the compression band may be removable from the compression garment. In another aspect, the compression assembly is integrated within the garment without the compression band. The compression garment may be disposed around the hip area of the user, i.e., from the lower abdomen and/or lower back to the upper part of the legs of the user. The compression band may be disposed on or within the compression garment, such that both may act as a unitary component.

The compression assembly may provide two stages of compression. A first stage achieves an initial “snug fit” by at least partially tightening the compression band and/or compression garment. Alternatively, the first stage of compression may also be achieved by an inherent compression of the compression garment and/or compression band itself, i.e., without any manual tightening. A second stage allows the user to selectively tighten the compression band to a different, and/or more customized compression force or level. The level of compression associated with the second compression stage may be higher than the level of compression associated with the first compression stage, but this is not strictly necessary as it may be lower. As such, the second level of compression may provide the user with controllable levels of pressure treatment.

A first compression mechanism may be disposed on the compression garment and/or the compression band to achieve the first stage of compression. The first compression mechanism or first tensioning device may incorporate various means such as snaps, Velcro, hooks, buttons, elastic mechanisms, e.g., Spandex, etc., or related mechanisms, to achieve and maintain the first stage of compression. The first compression mechanism may also comprise a release mechanism for the user to reduce the level of compression of the compression band and/or compression garment. Alternatively, and as briefly mentioned above, the compression band and/or compression garment may be provided with an inherent level of compression force that automatically achieves a snug fit, which provides the first level of compression.

A second compression mechanism may also be disposed on the compression band. The second compression mechanism generally comprises a tensioning device such as a rotating compression adjustment knob, i.e., a dial or similar mechanism, operatively configured with a tensioning structure or element such as, but not limited to cabling, wiring or an inextensible tensioning component, e.g., a strap of material operatively configured with a tube winder, etc., that may be retracted to induce a compression force. The compression adjustment knob of the tensioning device may be positioned along the sides of the compression band, but also in other locations such as the front or back thereof, including at an offset relation from the guide.

The second compression mechanism generally comprises tensioning elements comprising wiring or generally an inextensible material, which may be disposed on the inside of a guide and disposed around the perimeter of the compression band and/or compression garment according to the pattern of the guide. In a particular aspect, the guide may generally define two, or more, parallel lines disposed around the perimeter of the compression band and/or compression garment. The wiring or inextensible material should slide within the guide and should be sufficiently flexible to conform to the shape and/or arrangement of the guide. The wiring itself is generally connected to the compression adjustment knob or other tension adjusting device, which for simplicity is herein referred to as a compression adjustment knob.

As such, turning or rotating the compression adjustment knob of the tensioning device, results in coiling of the wiring, e.g., within a rotating drum, and shortens the length of the wiring within the guide. The result is a tightening of the compression band and/or compression garment. As the knob dial tightens the wiring loop and/or compression band/garment, a compression force increases around the treatment area of the user. Turning the compression adjustment knob in reverse direction, may lower the compression level. In addition, the tensioning device may include a slidable adjustable base. Accordingly, the position of the tensioning device may be adjusted on the compression band and/or compression garment relative to the tensioning elements to provide macro adjustment of compression. The compression adjustment knob may also be provided with a release mechanism, i.e., a button, switch, pop-up feature, or related mechanism or mechanisms to selectively and/or quickly lower the compression level.

The inventive compression assembly may also comprise a compression adjustment knob having multiple settings of adjustment to enable different compression increments. Accordingly, the compression adjustment knob can be configured to provide multiple stages of compression. For example, one mode of the compression adjustment knob may allow for large compression increments, whereas another mode may allow for smaller adjustments to “fine tune” the compression level.

In addition, the compression assembly may be operated pneumatically. For example, the tensioning element may include one or more chambers disposed around the perimeter of the compression band and/or compression garment that may be selectively filled with air, fluid, or other gas and pressurized to provide the compression.

A benefit would be further realized if such a compression assembly would also provide effective compression around an intended area of treatment. A further benefit would be realized by providing a compression assembly that can address the aforementioned needs through an integrated system comprising a compression garment and a compression band that is easy to position and remove. Yet a further benefit would be provided if such an assembly would comprise in some embodiments a compression band that could be removable from the garment, and in other embodiments a compression that is integrated into the garment. An even further benefit would be provided with a compression assembly that would allow a user to adjust the level of compression conveniently and accurately in multiple stages according to their desired level of tightness and therapeutic pressure.

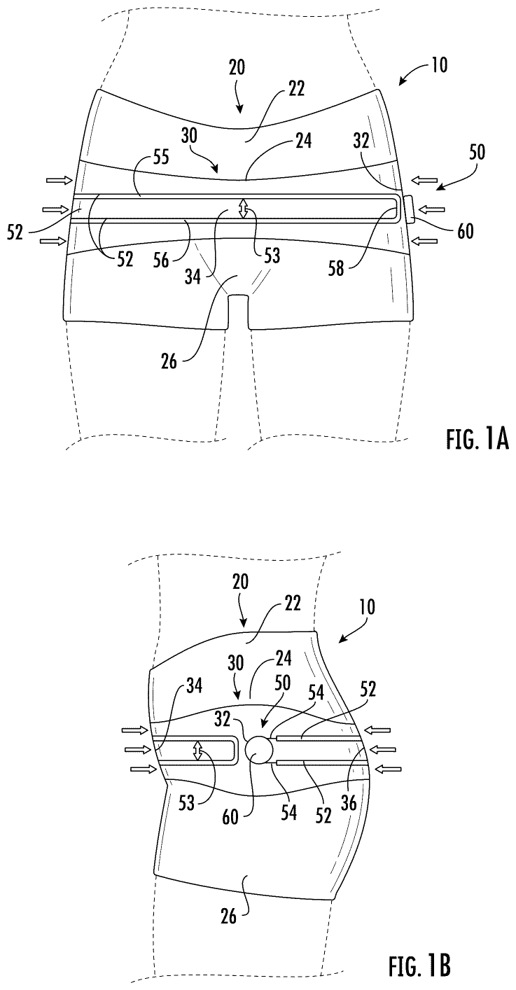

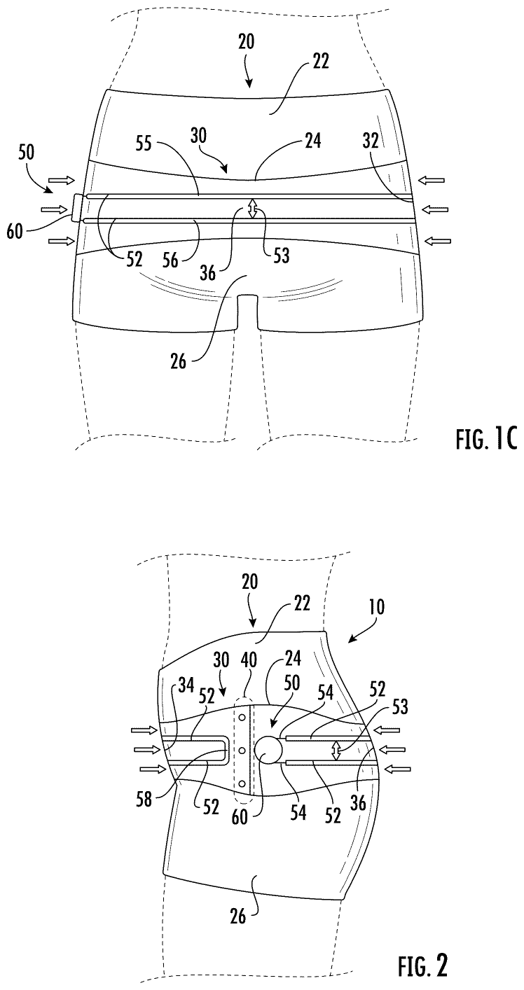

Referring now to FIGS. 1A, 1B, and 1C, the compression assembly is generally designated 10. It is within the scope of the present invention that the compression assembly 10 be user-adjustable and discrete. The compression assembly 10 may also generally provide for at least two stages of compression as discussed above. However, it is within the scope of the present invention that certain aspects of the compression assembly 10 only provide for one stage of compression as will be defined herein.

As an illustrative example, the inventive compression assembly 10 may be used to alleviate discomfort associated with a predetermined physical or medical condition such as hip pain, sciatica pain, menstrual pain or cramps, back pain, leg pain, etc.

The compression assembly 10 generally comprises a compression band 30 disposed or otherwise integrated into a compression garment 20. In alternative embodiments, a compression band 30 may be removably disposed onto or within the compression garment 20. In another aspect, the compression assembly and associated components are integrated within the garment and without the compression band 30. However, the compression band 30 and the compression garment 20 may collectively define a unitary component that can be worn by a user as a discrete garment piece or unit. That is, the compression band 30 may be disposed, connected, or attached to the compression garment 20 by means of sewing, gluing, clipping, adhesives, adhesive bands, mechanical attachments, etc.

The compression assembly 10 according to the present invention may be disposed around the hip area of the user, i.e., and may be positioned below the waist area and may extend to the upper leg area. As such, the compression garment 20 and/or compression band 30 may substantially create and/or distribute an inward pressure around the hip area of the user, including around the front or lower abdomen area, around the sides or hip area, and/or around the lower back area. The compression garment 20 and/or compression band 30 may generally seek to achieve a substantially uniform pressure distribution around the perimeter of the intended area of the user. However, as will be explained below, certain embodiments of the inventive compression assembly 10 may be configured to provide increased or targeted compression in certain zones or areas.

For example, such compression forces are represented by the arrows pointing toward the body of user. As mentioned above, the inventive compression assembly 10 contemplates a first and a second stage of compression. A first stage of compression may come from the compression garment 20 whereas a second stage of compression may come from a compression band 30. Alternatively, and as will be explained in more detail hereinafter, the compression band 30 itself may provide for the two different stages or levels of compression.

The compression assembly 10 may cover and/or may provide compression to at least a portion of the intended area of treatment of the user, which may be associated with discomfort from a predetermined condition. Again, such intended area may be around the hip, lower abdomen and/or lower back area. An upper section 22 of the compression garment 20 may be disposed around or right below the waist area of the individual. A middle section 24 of the compression garment 20 may be disposed around or below the hip area, and a lower section 26 of the compression garment 20 may be disposed around or below the groin and/or buttock area of the user. It is contemplated that at least the compression garment 20 and/or the compression band 30 be sufficiently flexible to allow for the user to position them around the intended area of treatment and/or to make adjustments that can achieve an intended or comfortable fit.

The compression garment 20 and/or the compression band 30 may also be configured and dimensioned to allow for the user to perform any such movements as would be possible otherwise, for example, walking, jogging, running, sitting, kneeling, stretching, extending, flexing the knees, etc. Furthermore, it is contemplated that the compression garment 20 and the compression band 30 should act as a unitary component. That is, a movement or adjustment to the position of the compression garment 20 may generally result in a reciprocal and corresponding movement of the compression band 30. Additionally, the compression garment 20 and/or the compression band 30 may be configured and dimensioned to correspond to the approximate size of the user.

After an initial positioning of the compression assembly 10 relative to the body of the user, a first stage of compression generally aims to achieve a preliminary tightening or “snug fit” of the compression garment 20 relative to the body of the user. As used herein, a “snug fit” may be defined as a fit of the compression assembly 10, including the compression garment 20 and/or compression band 30, relative to the hip area of the user that is either comfortable, or otherwise near a tight fit, but not a completely tight or strong fit.

Such a first stage of compression may be enabled by mechanically shortening the length of the perimeter or circumference of the compression garment 20 or the compression band 30 as shown in FIG. 2. For example, snaps, Velcro, hooks, or another comparable adjustment mechanism 40 may be used by the wearer to make an initial or first stage adjustment of the length of the band 30. Here, it is contemplated that the adjustment mechanism 40 comprises a release mechanism that may be activated to at least partially, or fully, reduce the compression level of the compression band 30. Alternatively, a compression assembly 10 may be provided with either a compression garment 20 and/or a compression band 30 that may automatically exert an inherent compression force, i.e., substantially without a manual adjustment, to achieve a first stage of compression or “snug fit.”

The compression band 30 may comprise a second compression or tensioning device 50 disposed thereon. The second compression or tensioning device 50 may be adjacent to a guide 52 disposed on or integrated with the compression band 30, i.e., on the side that faces away from the body of the user. The guide 52 may be disposed or integrated with the compression band 30 by a variety of mechanisms, including sewing, gluing, snap on mechanisms, clips, belts, screws, connectors, thermal bonding, etc. Further, the guide 52 should be disposed in non-movable relation to the compression band 30 and/or compression garment 20.

However, during the first and/or second stage of compression, it is contemplated that the compression band 30 will be at least partially contracted. Accordingly, the guide 52 should be configured to conform to the shape of the compression band 30 during either stage of compression, and/or without any compression. For example, the guide 52 should conform to the shape of the compression band before providing any compression, i.e., after an initial positioning of the compression garment, and/or when the compression or tensioning mechanisms 40 and/or 50 are released and the compression band 30 returns to its initial shape.

As may be appreciated by those of ordinary skill in the art, a guide 52 may be disposed on the compression band 30 in an arrangement comprising a first line 55 and/or second line 56. Alternatively, it is contemplated that only a first line 55 be disposed on the compression band 30. If both a first line 55 and a second line 56 are provided, they are generally disposed in a spaced apart relation to one another, with their spacing distance being shown at 53. The spaced apart relation between lines 55 and 56 provides for at least a partially enhanced stability of the compression assembly 10 when activating a first stage and/or a second stage of compression as defined herein.

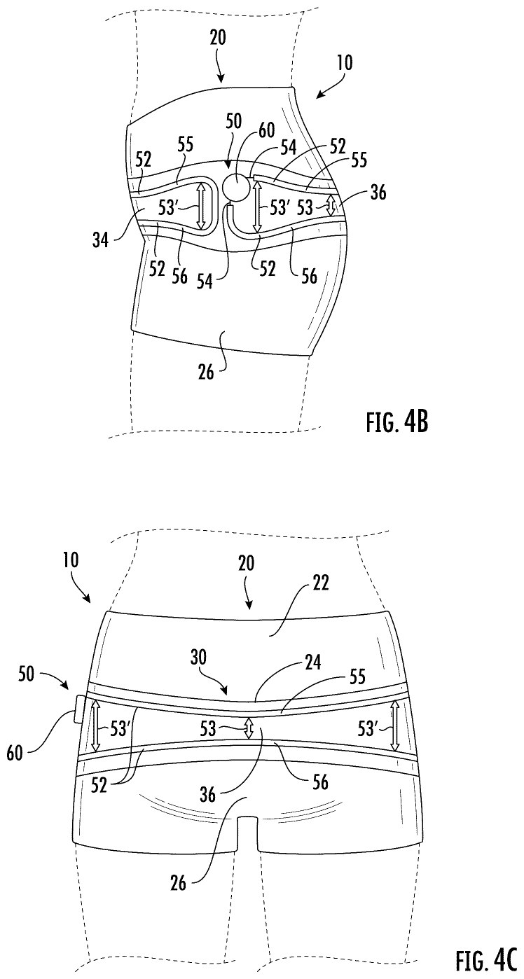

The spacing 53 between the lines 55 and/or 56 may be uniform around the perimeter of the band 30. However, as shown in FIGS. 4A, 4B, and 4C, the spacing 53′ may be non-uniform or otherwise non-parallel, e.g., longer around the hip area, including to allow for greater comfort and/or a more uniform compression. In addition, there are also other potential patterns of the guide 52, which may comprise a substantially serpentine, double-track arrangement. Also, although it is contemplated that the guide 52 comprises lines 55 and/or 56, this is not limiting, as additional lines, including four or more, may be disposed along the length of the compression band 30 or around specific intended areas thereof according to a predetermined path. This may be advantageous, for example, to more effectively target an intended compression area or zone around the user, thereby ensuring an overall better allocation of the tensioning pressure rather than in a wider area.

The guide 52 may substantially define a closed loop around the perimeter of the compression band 30. That is, the guide 52 may begin adjacent to a compression adjustment knob 60, e.g., of the second compression or tensioning device 50, which will be defined in more detail below, around a side 32 of the compression band 30 or garment 20. The compression adjustment knob 60 may be disposed on the compression band 30 in non-movable relation thereto. In another aspect, the tensioning device 50 may have a slidable base to allow for macro adjustment of the tensioning elements. The compression adjustment knob 60 may be disposed around the sides 32, front 34 or sides 32 of the compression band 30. The guide 52 may thus define a first line 55 substantially around the perimeter of the compression band 30. The guide 52 may also define a transition 58 adjacent to the knob 60, and may thereafter define a second line 56, also substantially around the perimeter of the compression band 30. Accordingly, both lines 55 and 56 of the second compression or tensioning device 50 may provide coverage to substantially the entirety of the perimeter of the body of the individual.

The second compression or tensioning device 50 may further comprise a tensioning element or tensioning element 54. Generally, the tensioning element 54 is operatively connected to the compression adjustment knob 60 of the tensioning device 50 and is operatively disposed on the inside of the guide 52. The tensioning element 54 may comprise a substantially inelastic tensioning element or otherwise an inextensible tensioning component, including without limitation, cabling or wiring disposed within the guide 52.

Additionally, the tensioning element 54 may comprise a non-extensible strap material cooperatively configured with a rotating mechanism, such as a tube winder with a fold-down handle. Further, the tensioning element 54 may comprise a variety of materials including, without limitation, plastic, silicone, metal, alloy, polymer, fabric, and/or combinations thereof. The tensioning element 54 and/or the guide 52, should be configured such that the tensioning element 54 may slidably move in a reciprocal direction within the guide 52.

Furthermore, the tensioning element 54 is configured such that it may at least partially conform to the shape of the guide 52, i.e., arrangement of lines 55/56 and/or transition 58. Additionally, the tensioning element 54 comprises a diameter that is at least smaller than the diameter of the guide. As such, it is contemplated that the tensioning element 54 will be operatively connected to the compression adjustment knob 60, e.g., at an upper portion thereof, and that it will enter the guide 52, i.e., on the first line 55.

The tensioning element 54 should also travel within the guide 52 around the back 36 of the compression band 30, pass through the transition 58 and should travel back on the inside of the guide 52, i.e., on the second line 56, towards the compression adjustment knob 60. As such the tensioning element 54 substantially spans around the perimeter of the compression band 30 and can provide compression around most of the hip area of the individual. The compression adjustment knob 60 may comprise an internal mechanism, i.e., a dial or a rotating drum for coiling the tensioning element 54.

In operation of the tensioning device 50, a rotation of the compression adjustment knob 60 results in a corresponding reduction of the effective length of the tensioning element 54, which may activate the first and/or second stage of compression. The compression adjustment knob 60 may comprise a rotating mechanism that may act as a coil to the tensioning element 54. As such, a rotation of the compression adjustment knob 60 in one direction may reduce the effective length of the tensioning element 54. As used herein, the “effective length” of the tensioning element 54 generally refers to the total length of the tensioning element 54 that is on the inside of the guide 52.

With a rotation of the compression adjustment knob 60 in a first direction, e.g., in the clockwise direction represented at 62, may result in a corresponding winding of the tensioning element 54 within the compression adjustment knob 60. Consequently, such rotation of the compression adjustment knob 60 will result in a corresponding reduction of the effective length of the tensioning element 54 within the guide 52. A reduction of the tensioning element 54 length within the guide 52 may also generally result in a corresponding shortening of the overall length of the compression band 30 and/or compression garment 20, thereby creating an inward pressure on the user.

Accordingly, a rotation of the compression adjustment knob 60 in an opposite direction may result in an increase of the effective length of the tensioning element 54, which at least partially reduces the overall level of compression of the compression band 30. Moreover, the compression adjustment knob 60 may comprise a release mechanism, i.e. a button that may be pushed, a switch that may be turned, magnets that can be separated, and/or a secondary rotation or click of the compression adjustment knob 60, that may be selectively activated to reduce the level compression of the compression band 30.

As explained above, the inventive compression assembly 10 may comprise a single compression or tension device 50 that can provide both a first and a second stage of compression. Accordingly, it may not always be necessary to provide a first compression or tensioning mechanism 40 or a compression garment 20 with an inherent compression force, although this may also be possible. For example, the garment assembly 20 alone may be configured to provide a first stage of compression whereas the compression band 30 may be configured to provide a second stage of compression, i.e., via tensioning device 50 and compression adjustment knob 60. In addition, the compression assembly and associated components may be integrated within the garment and without the compression band 30. Nonetheless, a single compression device 50 is also contemplated, which may provide a first and a second level of compression and which may comprise a compression adjustment knob 60 with two or more different settings.

For example, a plurality of different settings may be enabled by providing a rotating drum of the compression adjustment knob 60 with varying diameters. A plurality of different settings may also be enabled by providing a compression adjustment knob with predetermined rotational adjustment levels, i.e., a dial or ratcheting or clicking mechanism that rotates in specific amounts at a time according to user preferences.

Thus, a first setting may be enabled by pulling the compression adjustment knob 60 away from its initial position, i.e., by one click or snap away from the compression band 30. The first setting may be associated with an intended rotating drum diameter, and/or a predetermined rotational adjustment level, that may allow the compression adjustment knob 60 to reduce the effective length of the tensioning element 54 in increments associated with a first stage of compression.

Likewise, a second setting may be enabled by pulling the compression adjustment knob 60 away from its initial position. For example, this may be achieved by pulling the compression adjustment knob 60 away from the compression band 30 via a secondary click or snap. The second setting may be associated with an intended rotating drum diameter, and/or a predetermined rotational adjustment level, that may allow the compression adjustment knob 60 to reduce the effective length of the tensioning element 54 in increments associated with a first stage of compression.

As such, a user may selectively switch the settings of the compression adjustment knob 60 by pulling or pushing it, or alternatively, with another mechanical mechanism such as a button, a switch, a click, rachet, magnets, etc. Additionally, a release mechanism may be provided by pushing the compression adjustment knob 60 into the compression band 30 to a neutral position.

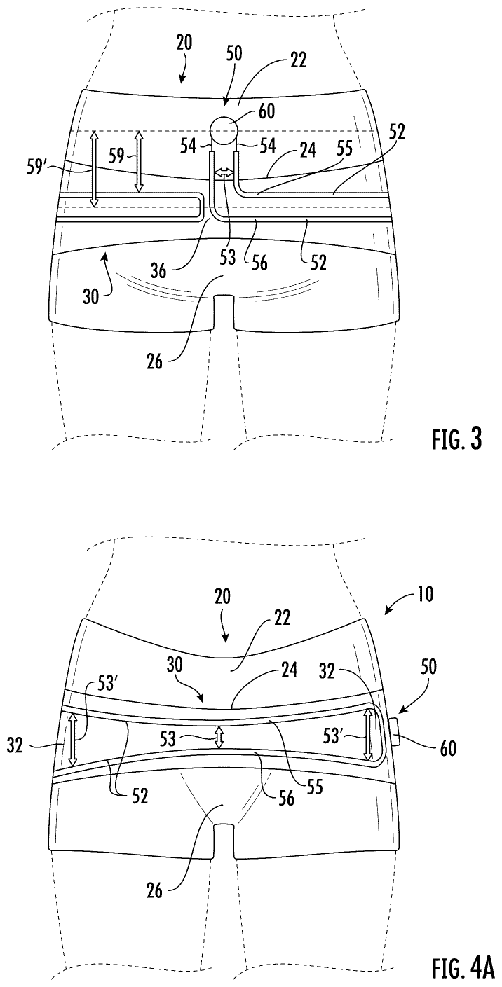

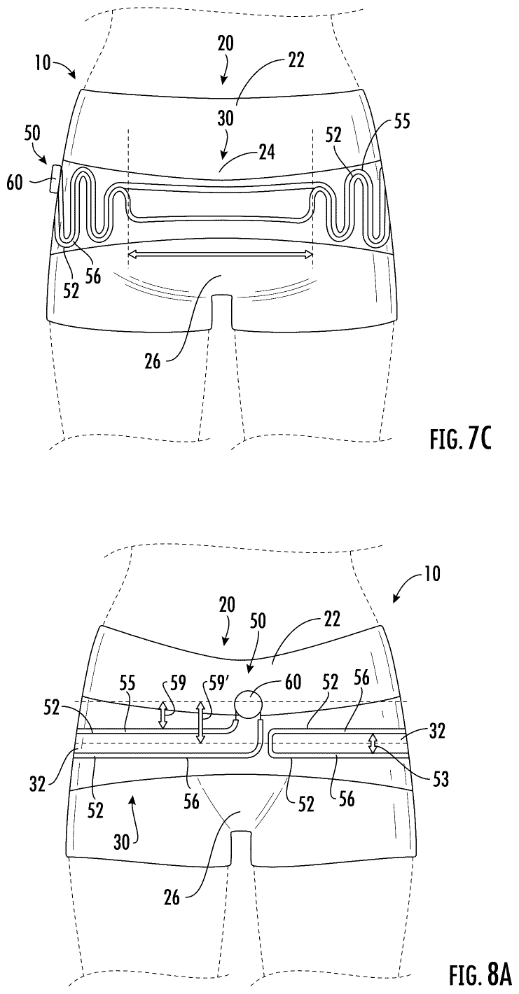

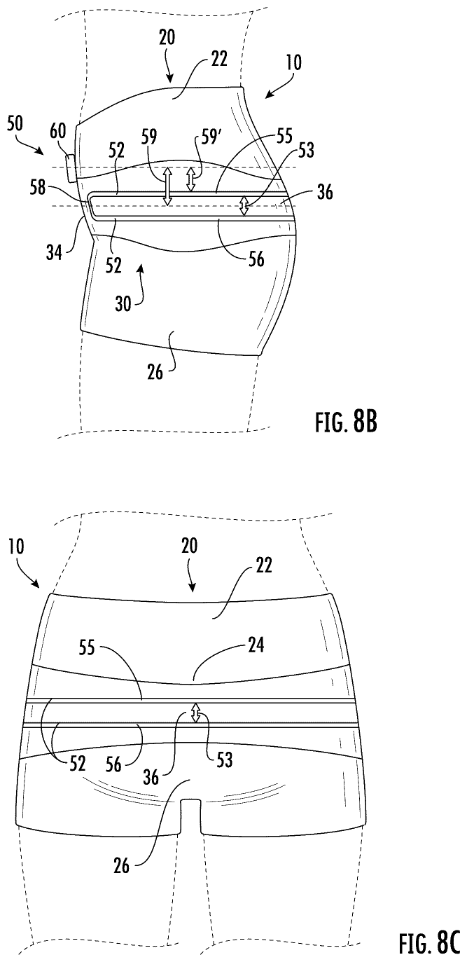

The compression adjustment knob 60 may be disposed in alternative locations on the compression band 30 and/or compression garment 20. As shown in FIG. 3, the compression adjustment knob 60 may be disposed on the back of the compression garment 20, for example, around the upper section 22 thereof. In contrast to FIG. 3, the compression adjustment knob 60 may be disposed on the front of the compression garment 20 around the upper section 22 as shown in FIG. 8A. The compression adjustment knob 60 may be disposed in an offset relation to the lines 55 and/or 56. For example, the compression adjustment knob 60 may be disposed at an offset distance 59 above the approximate middle of the spacing between the lines 55 and 56. As a further example, the compression adjustment knob 60 may be disposed at an offset distance 59′ above the first line 55.

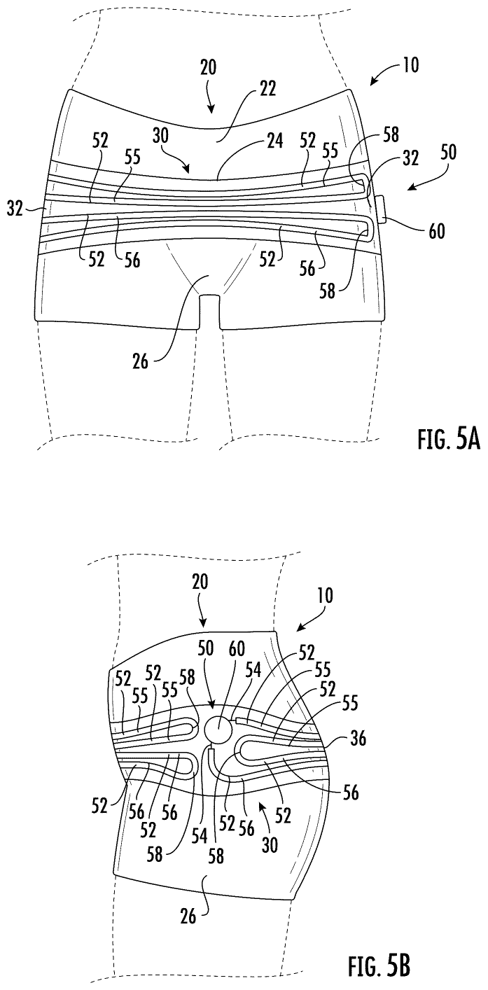

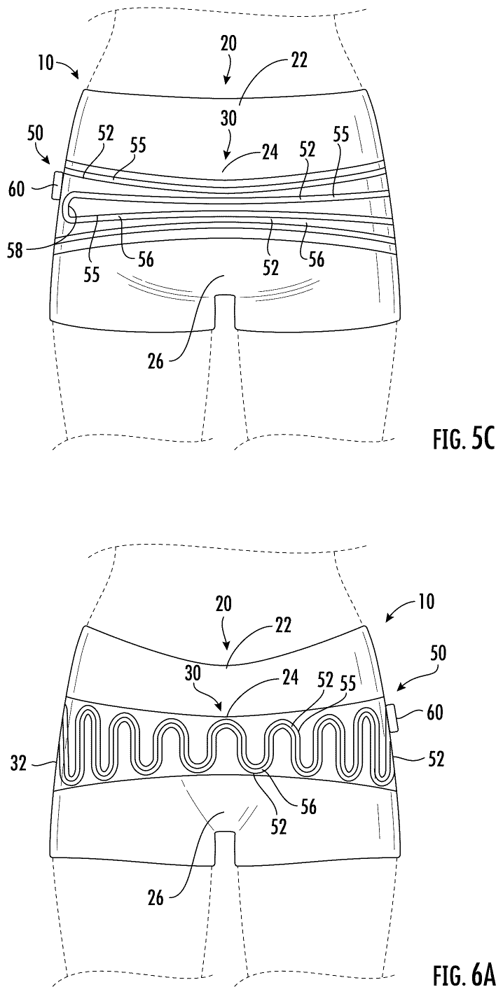

With reference now to FIGS. 5A, 5B, and 5C, the compression assembly 10 may be provided with additional lines of the tensioning element 54. For example, the compression assembly 10 may comprise one pair of lines 55 and a second pair of lines 56 spanning around the compression band 30. Here, it is contemplated that all pairs of lines be defined by the same tensioning element 54 and that they span around the circumference of the compression band 30 as many times as desired, i.e., making a transition(s) at desired areas, to define pairs of lines 55 and/or 56. As such, the compression band 30 may comprise four spaced apart lines 55 and/or 56 for added comfort, stability and/or effectiveness. Accordingly, the same tensioning element passes through three different transitions 58 around the compression band 30.

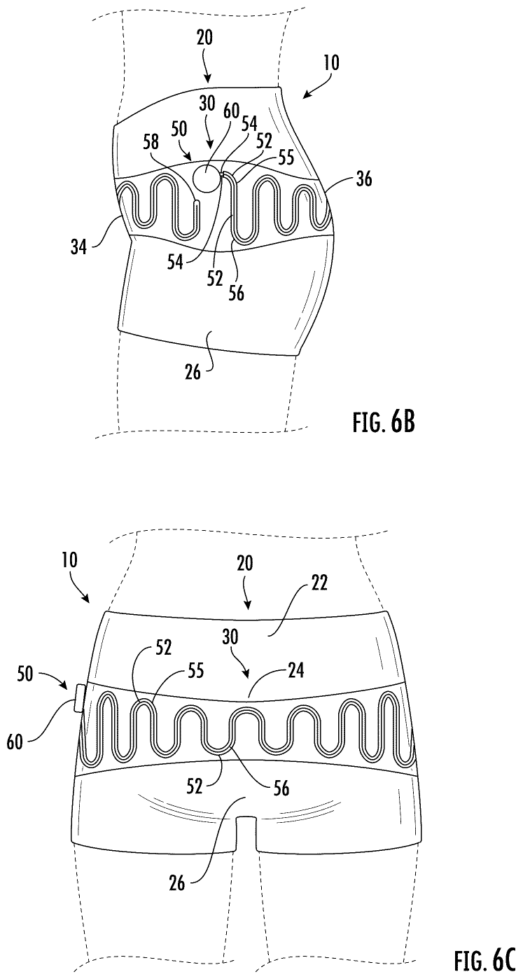

Referring now to FIGS. 6A, 6B, and 6C, the compression assembly 10 may be provided with various patterns of the lines of the tensioning element 54. For example, a tensioning element 54 may define a pair of adjacently disposed lines 55 and 56 disposed in a serpentine, or even a crisscross, arrangement to provide additional coverage to the intended treatment area or for added comfort. Such alternative patterns may also be provided to target specific sections of the intended treatment area. Accordingly, the tensioning element 54 may define one or two lines structured to provide only a tensioning force around the sides, i.e., around the hips, and not necessarily around the entire circumference of the compression band 30.

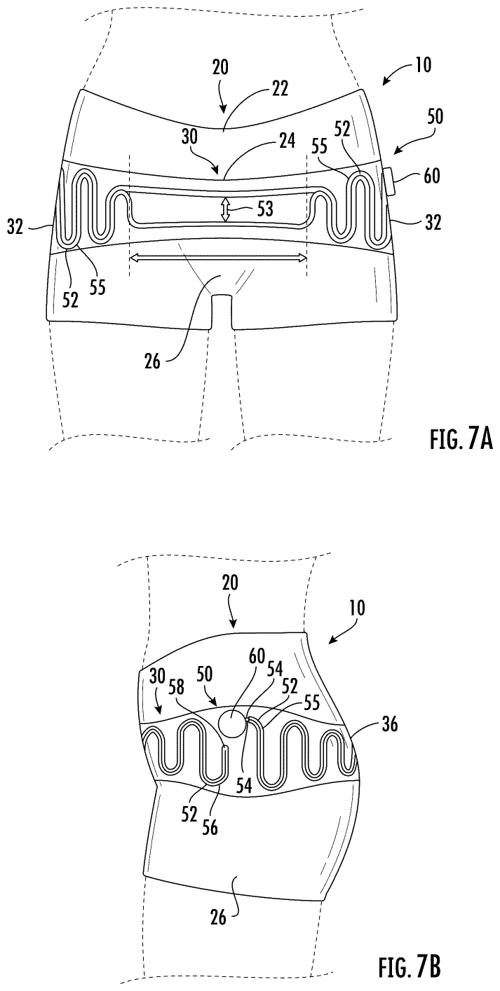

The compression assembly 10 may also comprise a tensioning element 54 having one pair of adjacently disposed lines 55 and 56 that collectively define a combination of a serpentine pattern around the sides of the hip area as shown in FIGS. 7A, 7B, and 7C.

In addition, a spaced apart relation of the lines 55 and 56 may be defined around the front area of the abdomen or lower abdomen as shown in FIGS. 8A, 8B, and 8C. The spaced apart relation of the lines 55 and 56 may be defined continuous around back area or lower back area.

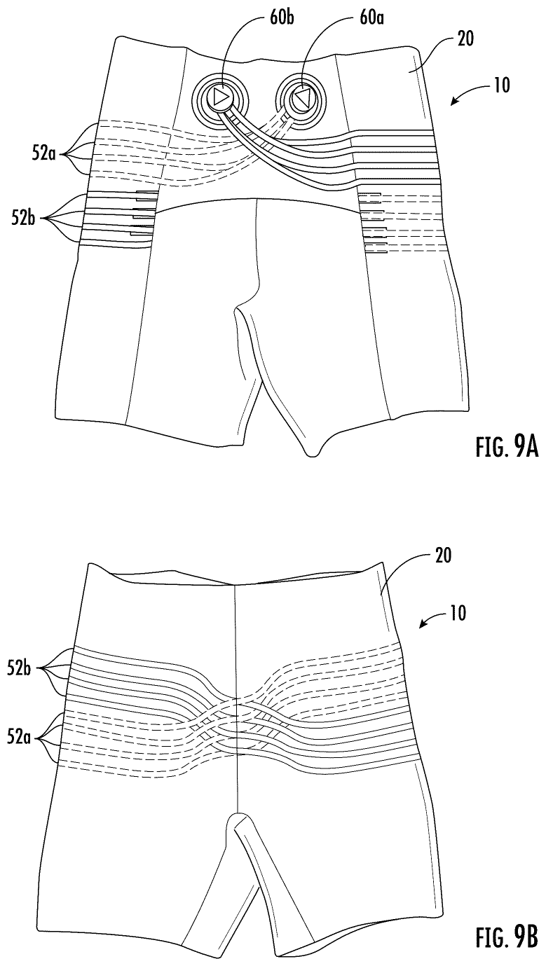

Referring now to FIGS. 9A and 9B, the compression assembly 10 includes a fabric material 20 having a waist opening on a first end and two leg openings on an opposing second end. The assembly also includes a first tensioning device 60a and a second tensioning device 60b secured to a portion of the fabric material. A plurality of guides 52a, 52b are integrated with the fabric material. The plurality of guides 52a, 52b run in a latitudinal direction along the fabric material between the waist opening and the two leg openings, and a plurality of tensioning elements 54 are positioned within the plurality of guides with each tensioning element having a distal end anchored to the fabric material and a proximal end secured to the respective tensioning device 60a, 60b. The distal end of the tensioning elements 54 also may be formed into an adjustable loop that is coupled back on itself.

The tensioning devices 60a, 60b are configured to selectively shorten a length of the respective tensioning elements 54 and result in applying compression to hip and back areas of a user wearing the compression assembly 10. In addition, the first set of guides 52a are positioned to run at least partially around a perimeter of the fabric material in a first direction, and the second set of guides 52b are positioned to run at least partially around the perimeter of the fabric material in an opposing second direction. Likewise, a first set of tensioning elements 54a are positioned to run through the first set of guides 52a in the first direction, and the second set of tensioning elements 54b are positioned to run through the second set of guides 52b in the opposing second direction.

The first tensioning device 60a is secured to respective proximal ends of the first set of tensioning elements 54a, and the second tensioning device 60b is secured to respective proximal ends of the second set of tensioning elements 54b.

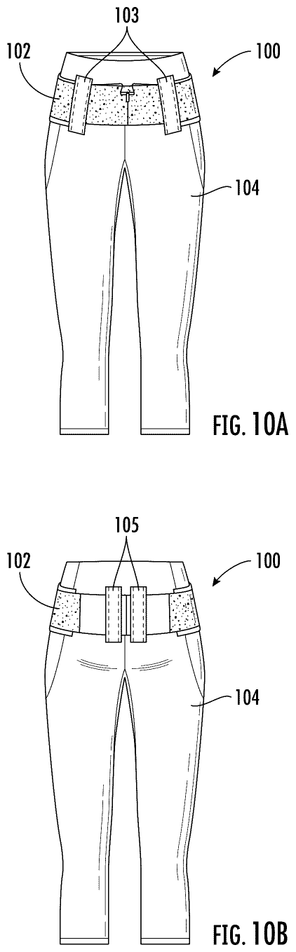

Referring now to FIGS. 10A and 10B, in another aspect the compression assembly 100 may be operated pneumatically. The compression assembly 100 includes a compression band 102 that may be secured to a garment 104 by front loops 103 and rear loops 105. The front loops and the rear loops 103, 105 are configured to maintain a proper positioning of the compression band 102 on the garment 104 when being worn by a user but they may not be required. As those of ordinary skill in the art can appreciate, the compression band 102 may also be used without the garment 104.

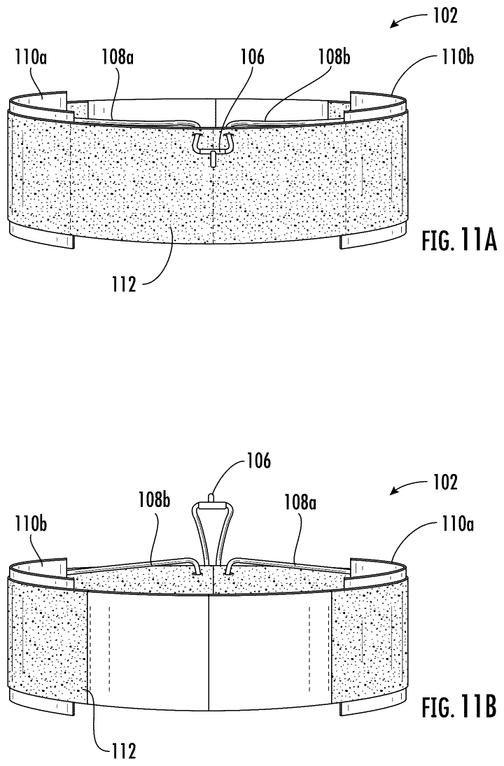

The compression band 102 may include integrated chambers configured to be filled with air under pressure to provide compression. The compression band 102 may includes a T-pipe 106 coupled to a pressurized air source that is in fluid communication with piping 108a, 108b as depicted in FIGS. 11A and 11B. The piping 108a, 108b may be coupled to a respective air chamber 110a, 110b. The air chambers 110a, 110b are held in place by fabric 112. The fabric 112 may be elastic and configured to apply compression to hip, lower back and/or lower abdomen areas of the user. The air chambers 110a, 110b are disposed around the perimeter of the compression band 102 and may be selectively filled with air, fluid, or other gas and be pressurized via piping 108a, 108b to provide compression.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

1. A compression assembly to alleviate physical discomfort, the compression assembly comprising:
a fabric material having a waist opening on a first end and two leg openings on an opposing second end;
a tensioning device secured to a portion of the fabric material;
a plurality of guides integrated with the fabric material, the plurality of guides running at least partially in a latitudinal direction along the fabric material between the waist opening and the two leg openings; and
a plurality of tensioning elements positioned within the plurality of guides, with each tensioning element having a distal end anchored and a proximal end secured to the tensioning device.

2. The compression assembly of claim 1, wherein the tensioning device is configured to selectively shorten a length of the plurality of tensioning elements and result in applying compression to hip and back areas of a user wearing the compression assembly.

3. The compression assembly of claim 1, wherein the plurality of guides comprises a first set of guides and a second set of guides, where the first set of guides are positioned to run at least partially around a perimeter of the fabric material in a first direction, and the second set of guides are positioned to run at least partially around the perimeter of the fabric material in an opposing second direction.

4. The compression assembly of claim 3, wherein the plurality of tensioning elements comprises a first set of tensioning elements and a second set of tensioning elements, where the first set of tensioning elements are positioned to run through the first set of guides in the first direction, and the second set of tensioning elements are positioned to run through the second set of guides in the opposing second direction.

5. The compression assembly of claim 4, wherein the tensioning device comprises a first tensioning device and a second tensioning device, the first tensioning device secured to respective proximal ends of the first set of tensioning elements, and the second tensioning device secured to respective proximal ends of the second set of tensioning elements.

6. The compression assembly of claim 1, wherein the fabric material is sized and shaped to apply compression to hip and back areas of a user in a first stage of initial compression.

7. The compression assembly of claim 6, wherein the tensioning device is configured to apply the compression to the hip and back areas of the user in a second stage of compression.

8. The compression assembly of claim 7, wherein the tensioning device comprises a rotating compression adjustment knob to selectively wind the plurality of tensioning elements thereon to maintain and apply a selected amount of compression in the second stage of compression.

9. The compression assembly of claim 8, wherein the tensioning device further comprises a ratcheting mechanism to maintain tension in the plurality of tensioning elements and a release mechanism that disengages the ratcheting mechanism when activated.

10. The compression assembly of claim 7, wherein the tensioning device comprises at least one of hook and loop fasteners, hooks, buckles, latches, and snaps.

11. The compression assembly of claim 1, wherein the plurality of tensioning elements comprises inelastic cables.

12. The compression assembly of claim 1, wherein the tensioning device has a slidable base configured for macro adjustment of the plurality of tensioning elements.

13. The compression assembly of claim 1, wherein the fabric material having a rear portion corresponding to a back of a user, a side portion corresponding to a hip of the user, and a front portion corresponding to an abdomen of the user, and the tensioning device being positioned on at least one of the rear portion, the side portion, and the front portion.

14. A compression assembly to alleviate physical discomfort, the compression assembly comprising:
a compression band having a waist opening and configured to be worn around a hip area by a user;
a tensioning device secured to a portion of the compression band;
a guide secured to the compression band and running at least partially in a latitudinal direction along the compression band; and
a tensioning element positioned within the guide and having a distal end anchored and a proximal end secured to the tensioning device.

15. The compression assembly of claim 14, wherein the tensioning device is configured to selectively shorten a length of the tensioning element and result in applying compression to a user wearing the compression assembly.

16. The compression assembly of claim 15, wherein the compression band is sized and shaped to apply compression to hip and back areas of a user in a first stage of initial compression.

17. The compression assembly of claim 16, wherein the tensioning device is configured to apply the compression to the hip and back areas of the user in a second stage of compression.

18. The compression assembly of claim 17, wherein the tensioning device comprises a rotating compression adjustment knob to selectively wind the tensioning element thereon to maintain and apply a selected amount of compression in the second stage of compression.

19. The compression assembly of claim 14, wherein the tensioning device has a slidable base to allow for macro adjustment of the tensioning element.

20. A method of applying compression to a hip area of a user using a compression assembly comprising a compression band having a tensioning device, a guide running at least partially in a latitudinal direction along the compression band, and a tensioning element positioned within the guide and having a distal end anchored and a proximal end secured to the tensioning device, the method comprising:
activating the tensioning device to selectively shorten a length of the tensioning element and result in applying compression to the user wearing the compression band.

21. The method of claim 19, the method further comprising activating the tensioning device to selectively lengthen the tensioning element and result in reducing an amount of compression being applied to the user wearing the compression band.
